(19) United States
(12) Patent Application Publication
(10) Pub. No.: US 2024/0041519 A1
(43) Pub. Date: Feb. 8, 2024

(54) INTRACORPOREAL MEMBER AND ELECTRODE

(71) Applicant: Japan Lifeline Co., Ltd., Tokyo (JP)

(72) Inventors: Toshiyuki IIJIMA, Tokyo (JP); Keisuke ADACHI, Tokyo (JP)

(21) Appl. No.: 18/350,335

(22) Filed: Jul. 11, 2023

(30) Foreign Application Priority Data
Aug. 8, 2022 (JP) 2022-126421

(51) Int. Cl. A61B 18/14

(52) U.S. Cl. CPC A61B 18/1492; A61B 2018/0022

(57) ABSTRACT

An intracorporeal member includes an elongate body having a distal end side, and electrodes disposed on the distal end side of the elongate body. At least the distal end side is to be inserted into a body. The electrode includes a first layer and a second layer arranged in a thickness direction of the electrode. At least a part of the second layer is located at a position farther away from the elongate body than the first layer. The first layer contains silver, gold, and carbon as main components, and contains carbon in a largest amount among silver, gold, and carbon. The second layer contains gold as a main component.

### CROSS REFERENCE TO RELATED APPLICATIONS

This application claims the benefit of priority to Japanese Patent Application Number 2022-126421 filed on Aug. 8, 2022. The entire contents of the above-identified application are hereby incorporated by reference.

### TECHNICAL FIELD

The present disclosure relates to an intracorporeal member and an electrode.

### BACKGROUND

A catheter is a member inserted into a body to perform diagnosis or treatment. For example, WO 2021/157100 discloses a balloon catheter including a balloon that can be inflated inside a body. In the balloon catheter, an electrode to which radio-frequency power is applied is disposed on a surface of the balloon.

### SUMMARY

When an electrode is provided on the surface of the balloon, the electrode is required to have stretchability (flexibility) so as to deform in response to deformation of the balloon. In addition, the balloon is required to be adhesive so that the electrode does not become detached from the balloon. Naturally, conductivity is also required. Further, since the electrode is to be inserted into the body, biological safety (low toxicity) is also required. The electrode is also required to have the above performance in a case where the electrode is directly provided on a shaft of a catheter or in a case where the electrode is provided on another intracorporeal member such as a medical needle.

The present disclosure has been made in view of such circumstances, and an object thereof is to provide a technique of improving the performance of an electrode to be inserted into a body.

An aspect of the present disclosure is an intracorporeal member. The intracorporeal member includes an elongate body having a distal end side, and an electrode disposed on the distal end side of the elongate body. At least the distal end side is to be inserted into a body. The electrode includes a first layer and a second layer arranged in a thickness direction of the electrode. At least a part of the second layer is located at a position farther away from the elongate body than the first layer. The first layer contains silver, gold, and carbon as main components, and contains carbon in a largest amount among silver, gold, and carbon. The second layer contains gold as a main component.

Another aspect of the present disclosure is an electrode for an intracorporeal member including an elongate body having a distal end side. At least the distal end side is configured to be inserted into a body. The electrode is disposed on the distal end side of the elongate body. The electrode includes a first layer and a second layer arranged in a thickness direction of the electrode. At least a part of the second layer is located at a position farther away from the elongate body than the first layer. The first layer contains silver, gold, and carbon as main components, and contains carbon in a largest amount among silver, gold, and carbon. The second layer contains gold as a main component.

Any combination of the above components and conversions of expressions of the present disclosure between a method, a device, a system, and the like are also effective as aspects of the present disclosure.

According to the present disclosure, the performance of an electrode to be inserted into a body can be improved.

### BRIEF DESCRIPTION OF DRAWINGS

FIG. 1 is a side view of an intracorporeal member according to an embodiment.

FIG. 2 is a cross-sectional view taken along line A-A in FIG. 1.

FIG. 3 is an enlarged cross-sectional view of a part of an electrode.

FIG. 4 is a graph showing a composition distribution of the electrode.

FIG. 5 is an enlarged cross-sectional view of an end portion of the electrode.

### DESCRIPTION OF EMBODIMENTS

The present disclosure will be described below based on preferred embodiments with reference to the drawings. The embodiments are illustrative and are not intended to limit the present disclosure. Not all features or combinations of the features described in the embodiments are essential to the present disclosure. The same or similar components, members, and processing operations illustrated in the drawings are denoted by the same reference numerals, and redundant descriptions are omitted as appropriate. The scales and shapes of the components illustrated in each drawing are set for convenience to facilitate the explanation and should not be construed in a limited manner unless otherwise specified. When the terms “first”, “second”, and the like are used in the specification or claims, these terms do not signify any order or importance unless otherwise specified and are used for distinguishing one configuration from other configurations. Furthermore, some of the members that are not critical in describing the embodiments in the drawings are omitted in the drawings.

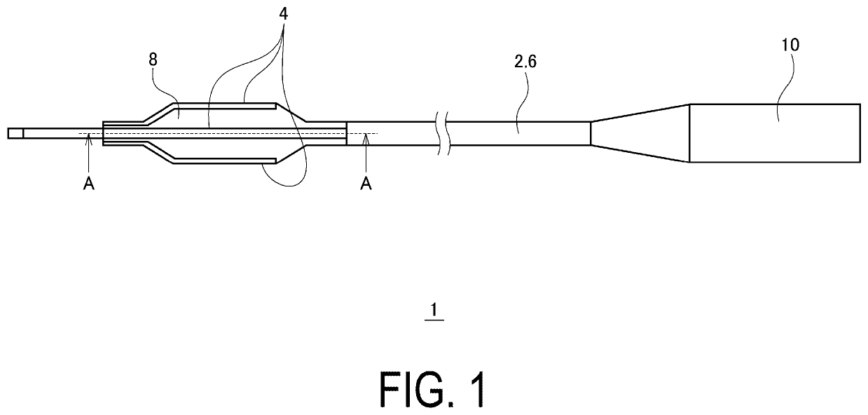

FIG. 1 is a side view of an intracorporeal member 1 according to an embodiment. The intracorporeal member 1 includes an elongate body 2 (rod body) having a distal end side, and electrodes 4 disposed on the distal end side of the elongate body 2. At least the distal end side is to be inserted into a body. The term “elongate” in the present embodiment means that a first length in a longitudinal direction is longer than a second length in a direction perpendicular to the longitudinal direction. For example, the ratio of the first length to the second length (first length/second length) is 5 or more. The intracorporeal member 1 of the present embodiment is, for example, a balloon catheter used for ablation. The balloon catheter is inserted into, for example, a tubular organ in the body, such as a blood vessel, the trachea, digestive tract, common bile duct, and pancreatic ductus; a connection portion between any of these; and a hole or tube formed in the body for testing or treatment, and is used for expanding or operating on a target site.

The balloon catheter serving as the intracorporeal member 1 includes a shaft 6, a balloon 8, electrodes 4, and a handle 10. The shaft 6 is configured as an elongate tubular body having flexibility. The shaft 6 is configured of a known flexible material including a resin, such as a polyolefin or a polyamide. The shaft 6 corresponds to the elongate body 2. The first length of the shaft 6 is, for example, from 600 mm to 1800 mm.

The balloon 8 is provided on a distal end (distal tip) side of the shaft 6. The balloon 8 is preferably attached at the distal end side relative to a half-length position of the entire length of the shaft 6. Hereinafter, a side on which the balloon catheter or the balloon 8 of the shaft 6 is provided is simply referred to as a “distal end side”, and the opposite (proximal tip) side is simply referred to as a “proximal end side”. The balloon 8 can be inflated by a fluid such as a contrast agent or saline supplied from the proximal end side of the shaft 6. The balloon 8 is configured to contract when the fluid is discharged. FIG. 1 illustrates the balloon 8 in an inflated state. The balloon 8 is configured of a known flexible material including a resin, such as a polyolefin or a polyamide.

The electrodes 4 are provided on a surface of the balloon 8. Each electrode 4 is configured of, for example, metal thin films layered on an outer surface of the balloon 8. In the present embodiment, the electrode 4 is formed in a strip shape extending in an axial direction of the shaft 6 (a direction in which the axis of the shaft 6 extends, or the longitudinal direction of the shaft 6), and a plurality of the electrodes are provided on the surface of the balloon 8. Each electrode 4 is connected to an external power supply (not illustrated) via a lead wire (not illustrated) inserted into the shaft 6, or laid on an outer surface of the shaft 6. When electric power is supplied from the external power supply to the electrode 4, the electrode 4 generates heat by Joule heating. The structure of the electrode 4 will be described in detail later.

The handle 10 is provided on the proximal end side of the shaft 6. The shaft 6 is inserted into the body from the distal end side. Accordingly, the balloon 8 and the electrodes 4 are sent into the body. The handle 10 is disposed outside the body and is held or manipulated by a practitioner.

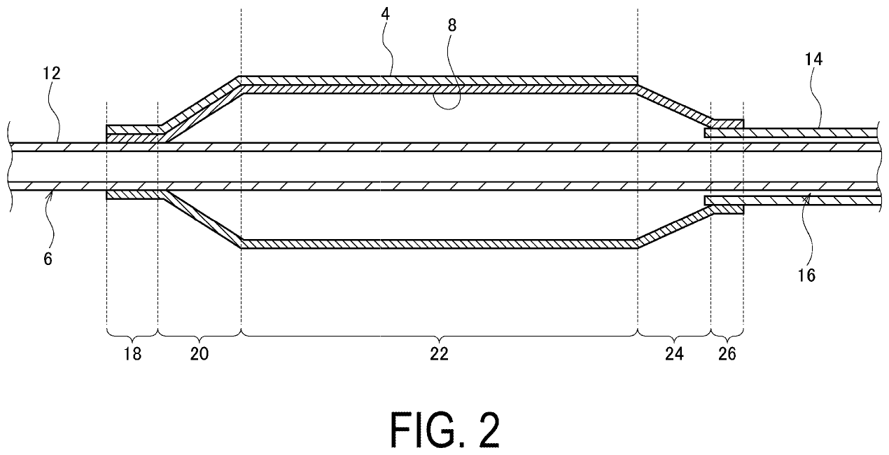

FIG. 2 is a cross-sectional view taken along line A-A in FIG. 1. As illustrated in FIG. 2, the shaft 6 includes an inner shaft 12 formed in a cylindrical shape and an outer shaft 14 formed in a cylindrical shape. The inner shaft 12 is inserted into the outer shaft 14. A distal end side of the inner shaft 12 protrudes from a distal end side of the outer shaft 14. The balloon 8 is disposed on this protruding portion. A balloon inflation lumen 16 is provided between an outer peripheral surface of the inner shaft 12 and an inner peripheral surface of the outer shaft 14. The balloon inflation lumen 16 functions as a passage for the fluid that inflates the balloon 8. One end of the balloon inflation lumen 16 is connected to an internal space inside the balloon 8. The other end of the balloon inflation lumen 16 extends to the handle 10.

The balloon 8 in the inflated state is divided into a distal-end-side neck portion 18, a distal-end-side tapered portion 20, an intermediate portion 22, a proximal-end-side tapered portion 24, and a proximal-end-side neck portion 26 from the distal end side toward the proximal end side. The distal-end-side neck portion 18 is fixed to the outer peripheral surface of the inner shaft 12. The distal-end-side tapered portion 20 increases in diameter from the distal-end-side neck portion 18 toward the proximal end side, and is connected to the intermediate portion 22. The intermediate portion 22 has substantially the same diameter over its entire length. The proximal-end-side tapered portion 24 decreases in diameter from the intermediate portion 22 toward the proximal end side, and is connected to the proximal-end-side neck portion 26. The proximal-end-side neck portion 26 is fixed to an outer peripheral surface of the outer shaft 14. Each electrode 4 extends from the distal-end-side neck portion 18 to the intermediate portion 22. Note that each of the electrodes 4 may have distal end sides, such as portions extending on the distal-end-side neck portion 18, connected to one another, thus allowing the respective electrodes 4 to be integrally formed.

In FIG. 2, the cross-sectional shapes of the respective portions are linear, with the distal-end-side tapered portion 20 being inclined relative to the distal-end-side neck portion 18 and the intermediate portion 22, and the proximal-end-side tapered portion 24 being inclined relative to the intermediate portion 22 and the proximal-end-side neck portion 26. Therefore, boundaries between the respective neck portions and the respective tapered portions and boundaries between the intermediate portion 22 and the respective tapered portions are clear. However, no limitation is intended, and the respective portions may be curved, making the boundaries between the respective portions unclear.

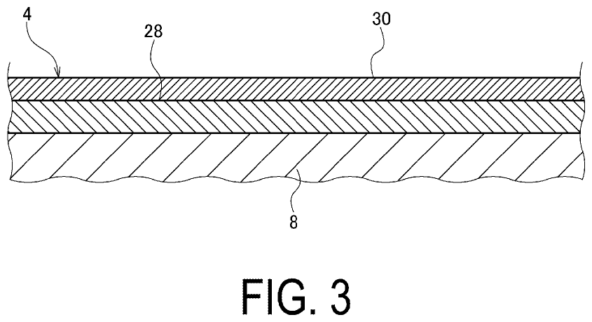

Next, the structure of the electrode 4 will be described in detail. FIG. 3 is an enlarged cross-sectional view of a part of the electrode 4. As illustrated in FIG. 3, the electrode 4 includes a first layer 28 and a second layer 30 arranged in a thickness direction of the electrode 4. At least a partial region of the second layer 30 is located at a position farther away from the balloon 8 (the elongate body 2 and the shaft 6) than the first layer 28. The first layer 28 of the present embodiment is in contact with the surface of the balloon 8, and extends across the surface. The second layer 30 is located on an outer surface of the electrode 4, is in contact with the first layer 28, and extends across the first layer 28. The second layer 30 is located at a position farther away from the balloon 8 than the first layer 28, excluding an end portion to be described later.

The first layer 28 need not be in direct contact with the balloon 8, and the second layer 30 need not be in direct contact with the first layer 28. In addition, another layer may be provided outside of the second layer 30. Further, for example, when the electrode 4 is divided into three equal parts in the thickness direction, a layer in contact with the balloon 8 may be defined as the first layer 28, and a layer including the outer surface of the electrode 4 may be defined as the second layer 30. The thickness of the first layer 28 is, for example, from 1 μm to 150 μm. The thickness of the second layer 30 is, for example, from 1 μm to 10 μm. As another example, the thickness of the first layer 28 is greater than the thickness of the second layer 30 by 10 μm or more.

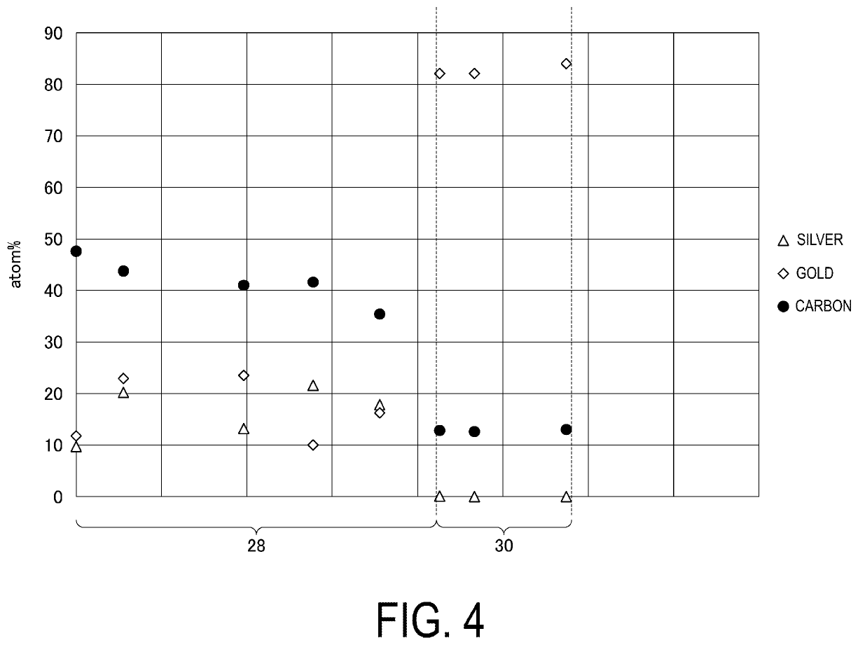

FIG. 4 is a graph showing a composition distribution of the electrode 4. The horizontal axis represents distance from the balloon 8, where an interface between the balloon 8 and the first layer 28 is 0. The vertical axis represents a ratio of each atom of gold (Au), silver (Ag), and carbon (C) to all components in each layer. The graph shown in FIG. 4 can be obtained as follows. Specifically, the electrode 4 is cut in the thickness direction. The cut surface is polished by a cross-section polisher to obtain an observation surface. Using an energy dispersive X-ray spectroscopy device (EDS) attached to a scanning electron microscope (SEM), the observation surface is irradiated at a plurality of portions with an electron beam. Thus, components of the portion irradiated with the electron beam and the contents thereof are measured. As a result, FIG. 4 is obtained. Nitrogen (N) and oxygen (O) are also detected in the measurement using the EDS but are not shown in FIG. 4.

As shown in FIG. 4, the first layer 28 contains silver, gold, and carbon as main components. The first layer 28 contains carbon in the largest amount among silver, gold, and carbon. Preferably, the amount of carbon is the largest among the amounts of all components contained in the first layer 28. The phrase “contains silver, gold, and carbon as main components” in the present embodiment means that the total of the contents of silver, gold, and carbon relative to all components constituting the first layer 28 is 50 atom % or more, preferably 70 atom % or more. When the total of the contents of silver, gold, and carbon is 50 atom % or more, the content of each of silver, gold, and carbon is preferably less than 50 atom %. When the total of the contents of silver, gold, and carbon is 70 atom % or more, the content of each silver, gold, and carbon is preferably less than 70 atom %.

On the other hand, the second layer 30 contains gold as a main component. The phrase “contains gold as a main component” in the present embodiment means that the content of gold is 50 atom % or more, preferably 70 atom % or more, relative to all components constituting the second layer 30. The content of each component in each layer is an average value of the content at a plurality of arbitrary measurement points shifted in the thickness direction and/or planar direction of each of the layers.

The content of silver in the first layer 28 is, for example, from 10 atom % to 30 atom %. The content of gold in the first layer 28 is, for example, from 10 atom % to 25 atom %. The content of carbon in the first layer 28 is, for example, from 30 atom % to less than 50 atom %.

The content of silver in the second layer 30 is, for example, from 0 atom % to 1 atom %. The content of gold in the second layer 30 is, for example, from 80 atom % to 99 atom %. The content of carbon in the second layer 30 is, for example, from 1 atom % to 20 atom %.

Preferably, the second layer 30 has a lower content of at least one of silver and carbon than the first layer 28. In the second layer 30 of the present embodiment, the contents of both silver and carbon are lower than the contents of silver and carbon in the first layer 28. The difference between the content of silver in the first layer 28 and the content of silver in the second layer 30 is, for example, from 9 atom % to 30 atom %. The difference between the content of carbon in the first layer 28 and the content of carbon in the second layer 30 is, for example, from 10 atom % to 49 atom %. The difference between the content of gold in the first layer 28 and the content of gold in the second layer 30 is from 55 atom % to 89 atom %.

The electrode 4 including the first layer 28 and the second layer 30 can be obtained by, for example, applying a conductive ink for the first layer 28 to the surface of the balloon 8 to form the first layer 28, and then applying a conductive ink for the second layer 30 to a surface of the first layer 28 to form the second layer 30. Each conductive ink contains the metal components constituting each layer and a solvent.

In general, the constituent components of the electrode 4 include silver for imparting conductivity to the electrode 4 and a resin for imparting, to the electrode 4, stretchability and adhesiveness to the balloon 8. The carbon described above is derived from the resin. Increasing the amount of the resin is a way to make the electrode 4 more easily deform in response to the deformation of the balloon 8 and further suppress the electrode 4 from separating from the balloon 8. However, increasing the amount of the resin may decrease the conductivity of the electrode 4. When the conductivity of the electrode 4 decreases, the electrode 4 is likely to generate heat, and may cause damage to the balloon 8.

On the other hand, when the content of silver is increased to prioritize the conductivity of the electrode 4, the stretchability and adhesiveness of the electrode 4 may decrease. When a balloon catheter is manufactured, generally, the electrode 4 is formed while the balloon 8 is inflated, and then the balloon 8 is folded. For this reason, it is desirable to suppress the occurrence of cracks in the electrode 4 or separation of the electrode 4 from the balloon 8 when the balloon 8 is folded.

In addition, even when the stretchability, adhesiveness, and conductivity of the electrode 4 are ensured by adjusting the blended amounts of silver and the resin, there is a concern that the resin or silver may be released into the body and exhibit cytotoxicity. When the solvent of the conductive ink remains in the electrode 4 and is released into the body, the solvent may similarly exhibit cytotoxicity. Therefore, in addition to the stretchability, adhesiveness, and conductivity, it is also necessary to ensure the biological safety of the electrode 4. The stretchability, adhesiveness, conductivity, and safety of the electrode 4 also needs to be ensured when the electrode 4 is directly provided on a surface of the elongate body 2 including the shaft 6.

Meanwhile, the electrode 4 of the present embodiment includes the first layer 28 containing silver, gold, and carbon as main components and containing carbon in the largest amount among these three components, and the second layer 30 containing gold as a main component and covering the first layer 28. Since carbon in the first layer 28 is derived from the resin, the first layer 28 contains silver, gold, and the resin as main components.

When the first layer 28 contains silver, gold, and a resin as main components, and the proportion of the resin is higher than that of silver and gold, a more favorable balance among the stretchability, adhesiveness, and conductivity of the electrode 4 can be achieved. The first layer 28 contains not only silver but also gold as a conductive component. Because gold is more ductile than silver, a decrease in the stretchability can be suppressed while increasing the conductivity of the electrode 4.

In addition, since the first layer 28 is covered with the second layer 30 containing gold as a main component, dissolution of the resin, silver, and the residual solvent contained in the first layer 28 can be suppressed. With this configuration, the safety of the electrode 4 can be improved. The conductivity of the electrode 4 can also be improved. The second layer 30 of the present embodiment is located on the outer surface of the electrode 4. With this configuration, the safety of the electrode 4 can be further improved. The second layer 30 has a lower content of at least one of silver and carbon than the first layer 28. With this configuration, the safety of the electrode 4 can be further improved.

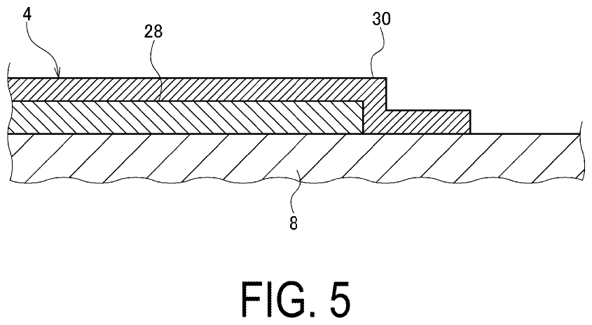

FIG. 5 is an enlarged cross-sectional view of an end portion of the electrode 4. As illustrated in FIG. 5, preferably, an end portion of the first layer 28 is covered with the second layer 30. The second layer 30 of the present embodiment extends to the outside of the first layer 28 in a direction along the surface of the balloon 8, and the end portion extending to the outside of the first layer 28 is in contact with the surface of the balloon 8. Therefore, both the main surface of the first layer 28 and end surfaces of the first layer 28 are covered with the second layer 30. When the entire first layer 28 is covered with the second layer 30, the safety of the electrode 4 can be further improved. The above-described structure can also be interpreted in that the first layer 28 includes a central portion containing silver, gold, and carbon as main components and the end portion containing gold as a main component.

The embodiment of the present disclosure has been described in detail. The embodiment described above is merely a specific example for carrying out the present disclosure. The content of the embodiment is not intended to limit the technical scope of the present disclosure. Various design changes such as alterations, additions, and deletions of components can be made within a scope that does not depart from the spirit of the present disclosure specified in the claims. A new embodiment with design changes has the effects of combined embodiments and variations thereof. In the embodiment described above, the content to which such design changes can be made has been emphasized with expressions such as “of the present embodiment” or “in the present embodiment”, but design changes are also possible for content not indicated by such an expression. Any combination of components included in each embodiment is also effective as an aspect of the present disclosure. Hatching in sections of the drawings does not limit the material of the hatched object.

The embodiment may be identified by the items described below.

### [Item 1]

An intracorporeal member (1), including:

an elongate body (2) having a distal end side; and

an electrode (4) disposed on the distal end side, wherein

at least the distal end side is inserted into a body,

the electrode (4) includes a first layer (28) and a second layer (30) arranged in a thickness direction of the electrode (4), at least a part of the second layer (30) being located at a position farther away from the elongate body (2) than the first layer (28),

the first layer (28) contains silver, gold, and carbon as main components, and contains carbon in a largest amount among silver, gold, and carbon, and

the second layer (30) contains gold as a main component.

### [Item 2]

The intracorporeal member (1) according to Item 1, in which

the elongate body (2) is a shaft (6) having a tubular shape and having flexibility, and

the intracorporeal member (1) is a catheter.

### [Item 3]

The intracorporeal member (1) according to Item 2, including:

a balloon (8) disposed on a distal end side of the shaft (6), the balloon (8) being inflatable by a fluid supplied from a proximal end side of the shaft (6), wherein

the electrode (4) is disposed on a surface of the balloon (8).

### [Item 4]

The intracorporeal member (1) according to Item 3, in which

the first layer (28) is in contact with the surface of the balloon (8); and

the second layer (30) is located on an outer surface of the electrode (4).

### [Item 5]

The intracorporeal member (1) according to any of Item 1 to Item 4, in which the second layer (30) has a lower content of at least one of silver and carbon than the first layer (28).

### [Item 6]

The intracorporeal member (1) according to any of Item 1 to Item 5, in which an end portion of the first layer (28) is covered with the second layer (30).

### [Item 7]

An electrode (4) for an intracorporeal member (1) including an elongate body (2) having a distal end side, at least the distal end side being configured to be inserted into a body, the electrode (4) being disposed on the distal end side, the electrode (4) including:

a first layer (28) and a second layer (30) arranged in a thickness direction of the electrode (4), at least a part of the second layer (30) being located at a position farther away from the elongate body (2) than the first layer (28), wherein

the first layer (28) contains silver, gold, and carbon as main components, and contains carbon in a largest amount among silver, gold, and carbon, and

the second layer (30) contains gold as a main component.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. An intracorporeal member, comprising:
an elongate body having a distal end side; and
an electrode disposed on the distal end side, wherein
at least the distal end side is to be inserted into a body,
the electrode includes a first layer and a second layer arranged in a thickness direction of the electrode, at least a part of the second layer being located at a position farther away from the elongate body than the first layer,
the first layer contains silver, gold, and carbon as main components, and contains carbon in a largest amount among silver, gold, and carbon, and
the second layer contains gold as a main component.

2. The intracorporeal member according to claim 1, wherein
the elongate body is a shaft having a tubular shape and having flexibility, and
the intracorporeal member is a catheter.

3. The intracorporeal member according to claim 2, comprising
a balloon disposed on a distal end side of the shaft, the balloon being inflatable by a fluid supplied from a proximal end side of the shaft, wherein
the electrode is disposed on a surface of the balloon.

4. The intracorporeal member according to claim 3, wherein
the first layer is in contact with the surface of the balloon, and
the second layer is located on an outer surface of the electrode.

5. The intracorporeal member according to claim 1, wherein
the second layer has a lower content of at least one of silver and carbon than the first layer.

6. The intracorporeal member according to claim 1, wherein
an end portion of the first layer is covered with the second layer.

7. An electrode for an intracorporeal member including an elongate body having a distal end side, at least the distal end side being configured to be inserted into a body, the electrode being disposed on the distal end side, the electrode comprising:
a first layer and a second layer arranged in a thickness direction of the electrode, at least a part of the second layer being located at a position farther away from the elongate body than the first layer, wherein
the first layer contains silver, gold, and carbon as main components, and contains carbon in a largest amount among silver, gold, and carbon, and
the second layer contains gold as a main component.
